(19) United States
(12) Patent Application Publication
(10) Pub. No.: US 2026/0276623 A1
(43) Pub. Date: Sep. 17, 2026

(54) SYSTEM AND METHOD WITH ARTIFICIAL INTELLIGENCE PATCH CLAMP FOR AUTOMATED RECORDING AND KINETICS ANALYSIS

(71) Applicant: City University of Hong Kong, Hong Kong (HK)

(72) Inventors: Wai Chiu King LAI, Hong Kong (HK); Shengjie YANG, Shenzhen (CN)

(21) Appl. No.: 19/076,999

(22) Filed: Mar. 11, 2025

(51) Int. Cl. G01N 33/487; G06N 3/0442; G06N 3/045; G06N 3/0464

(52) U.S. Cl. CPC G01N 33/48728; G06N 3/0442; G06N 3/045; G06N 3/0464

(57) ABSTRACT

An automated patch clamp system includes a computing platform, a cell dish, a pipette, a micromanipulator, a pressure regulator, recording electrodes, and a digitizer. The cell dish holds at least one cell in a physiological solution for electrophysiological measurement. The pipette interfaces with the cell membrane to establish a gigaseal, positioned by the micromanipulator based on computing platform control. The pressure regulator applies suction for gigaseal formation. One recording electrode is in the pipette, the other in the bath solution, detecting ionic currents. The digitizer converts detected signals into digital form. The computing platform processes these signals using an AI framework or machine learning model for ion channel kinetics analysis and anomaly detection, classifying ion channel types based on extracted electrophysiological features.

### TECHNICAL FIELD

The present invention relates to the field of artificial intelligence (AI), particularly to automated patch clamp systems and methods for real-time sealing condition monitoring, recording, and AI-driven electrophysiological data analysis.

### BACKGROUND

Patch clamp techniques, particularly automated systems, are widely employed to investigate electrophysiological properties, providing critical insights into the electrical behavior of individual ion channels. Ion channels play a fundamental role in various cellular functions, including signal transmission, excitability, and physiological responses to stimuli. As a result, patch clamp methodologies are extensively utilized in disciplines such as neuroscience, cardiology, pharmacology, and biophysics to study ion channel behavior under different conditions, including the effects of pharmaceutical compounds and other interventions. However, conventional patch clamp systems face significant limitations due to their reliance on manual control, inconsistent sealing conditions, and the absence of integrated data analysis tools.

Traditional patch clamp techniques require substantial operator expertise to determine and maintain the appropriate seal conditions necessary for gigaseal formation and high-quality recordings. This challenge arises from the inherent limitations of existing pipette-cell membrane deformation models, which fail to accurately predict and regulate the interaction between the pipette tip and the cell membrane. The unpredictable nature of the contact gap between the pipette and the membrane poses difficulties in controlling leakage currents and achieving a stable, high-resistance seal. Successful patch clamp recordings demand precise, rapid, and disturbance-resistant nanoscale manipulations to establish a robust electrical connection and enhance the signal-to-noise ratio.

Despite advancements in automation, existing automated patch clamp systems primarily replicate the lipid-glass sealing criteria and manipulation techniques of manual methods without incorporating adaptive optimization based on real-time sealing parameters. These systems lack the capability to dynamically assess and adjust sealing conditions on a case-by-case basis, leading to persistent challenges in gigaseal formation and recording stability. Consequently, there has been no significant improvement in gigaseal success rates or data acquisition yields compared to traditional manual approaches. The inability of current automated systems to enhance sealing precision and recording quality remains a critical barrier to achieving fully autonomous and high-throughput patch clamp experimentation.

Accordingly, there is a need for an advanced automated patch clamp system that integrates real-time sealing condition monitoring, adaptive optimization, and AI-driven data analysis.

### SUMMARY OF INVENTION

It is an objective of the present invention to provide a system and a method to solve the aforementioned technical problems.

This invention introduces an advanced automated patch clamp platform that integrates real-time sealing condition monitoring, adaptive control, and artificial intelligence-driven data analysis. By providing a unified solution for both data acquisition and ion channel behavior analysis, the system significantly enhances the precision, efficiency, and applicability of cellular electrophysiology studies across various research and diagnostic fields. Through the integration of a lipid-glass sealing strategy with real-time feedback, the system optimizes gigaseal formation, ensuring stable and high-quality electrophysiological recordings while automating data interpretation.

To achieve this, the system employs a quantifiable sealing model that translates lipid-glass deformation into real-time electrical signals, enabling precise adjustments to the pipette position based on calculated seal ratios derived from initial bath current or series resistance measurements.

Furthermore, the system features an artificial intelligence framework that leverages both machine learning and deep learning techniques for ion channel analysis. The artificial intelligence framework enables automated anomaly detection and multi-class classification, allowing for the identification of multiple ion channel types and their kinetic properties within a single recording, thus streamlining electrophysiological research and improving data reliability.

In accordance with a first aspect of the present invention, an automated patch clamp system is provided. The automated patch clamp system includes a computing platform, a cell dish, a pipette, a micromanipulator, a pressure regulator, a pair of recording electrodes, and a digitizer. The cell dish is configured to hold at least one cell in a physiological solution for electrophysiological measurement. The pipette is configured to interface with a cell membrane of the cell to establish a gigaseal. The micromanipulator is configured to position the pipette relative to the cell membrane based on control signals from the computing platform. The pressure regulator is coupled to the pipette and is configured to regulate a suction pressure applied for gigaseal formation. One of the recording electrodes is positioned within the pipette and another one is in a bath solution within the cell dish. The recording electrodes are configured to detect at least one ionic current flowing through ion channels of the cell. The digitizer is configured to convert the detected ionic current into digitized electrophysiological signals. The computing platform is further configured to process the digitized electrophysiological signals by executing an AI framework or a machine learning model for ion channel kinetics analysis and anomaly detection, classifying ion channel types based on electrophysiological features extracted from the ion channel kinetics analysis.

In accordance with a second aspect of the present invention, an automated patch clamp method is provided. The automated patch clamp method includes steps as follows: holding at least one cell in a physiological solution using a cell dish for electrophysiological measurement; interfacing with a cell membrane of the cell to establish a gigaseal using a pipette; positioning the pipette relative to the cell membrane using a micromanipulator based on control signals from a computing platform; coupling a pressure regulator to the pipette to regulate a suction pressure applied for gigaseal formation; detecting at least one ionic current flowing through ion channels of the cell using a pair of recording electrodes; converting the detected ionic current into digitized electrophysiological signals using a digitizer; and processing the digitized electrophysiological signals by executing an AI framework or a machine learning model, using the computing platform, for ion channel kinetics analysis and anomaly detection, thereby classifying ion channel types based on electrophysiological features extracted from the ion channel kinetics analysis.

By the configuration, the automated patch clamp system provided by the present invention combines recording acquisition with ion channel kinetics analysis in a single machine. The configuration integrates the pipette-cell membrane sealing mechanism with real-time electrical signal monitoring to develop a lipid-glass sealing strategy, providing tailored operating parameters and optimal sealing conditions for each test during gigaseal formation and recording.

Unlike existing systems, the platform of the present invention performs anomaly detection and ion channel kinetic analysis on the acquired recordings to extract electrophysiological properties, physiological functions, and cellular characteristics. Anomaly detection employs machine learning algorithms, such as K-nearest neighbors, to exclude data lacking physiological significance, including aberrant transient ion channel behaviors, failed recordings, and partially abnormal ion channel activity. The deep learning network, including a one-dimensional convolutional neural network and bidirectional long short-term memory, performs multi-class classification on action-based segmented recordings to characterize ion channel types, behaviors, and kinetics.

The provided features enable unattended patch clamp technology with an integrated measurement and analysis design, improving success rates and yields while reducing complexity and reliance on operator expertise. Additionally, real-time data collection using microscopes and digitizers enhances both the accuracy and effectiveness of measurements, making the provided system a superior option for modern patch clamp recording.

### BRIEF DESCRIPTION OF DRAWINGS

Embodiments of the invention are described in more details hereinafter with reference to the drawings, in which:

FIG. 1 shows a schematic diagram of a structure of an automated patch clamp system according to some embodiments of the present invention;

FIG. 2 illustrates a workflow of the automated patch clamp system detailing the process for conducting electrophysiological recordings and analyzing ion channel kinetics according to some embodiments of the present invention;

FIG. 3 illustrates a workflow of the machine learning-based anomaly detection of step S222 according to some embodiments of the present invention; and

FIG. 4 shows a neural network architecture of deep learning for multi-class classification of step S224 according to some embodiments of the present invention.

### DETAILED DESCRIPTION OF THE INVENTION

In the following description, automated patch clamp systems and methods for real-time sealing condition monitoring, recording, and AI-driven electrophysiological data analysis, and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

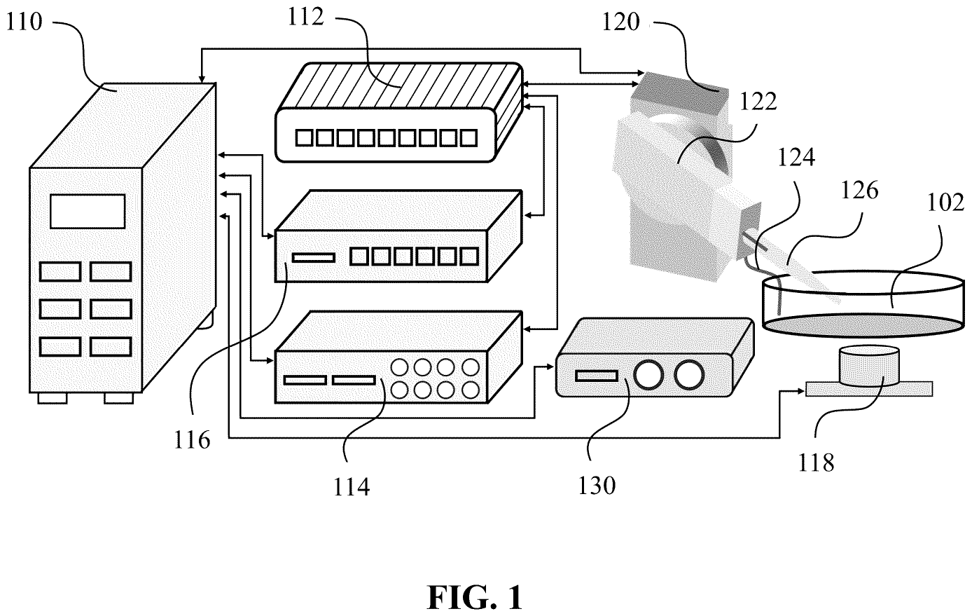

FIG. 1 shows a schematic diagram of a structure of an automated patch clamp system 100 according to some embodiments of the present invention. The automated patch clamp system 100 integrates hardware components with artificial intelligence (AI) model/module to perform a patch clamp process, achieving unattended electrophysiological measurements. The system 100 automates the patch clamp process, from gigaseal formation to ion channel kinetics analysis, offering efficiency and accuracy.

The automated patch clamp system 100 includes a cell dish 102, a computing platform 110, an amplifier 112, a digitizer 114, a data acquisition system (DAQ) system 116, a microscope 118, a micromanipulator 120, a headstage 122, a pair of recording electrodes 124, a pipette 126, a pressure regulator 130.

The cell dish 102 holds one or more cells for electrophysiological monitoring and investigation of the patch clamp process. The cells are submerged in a physiological solution that simulates their natural environment, providing suitable conditions for measuring ion channel behavior in their native state.

The computing platform 110 is a central unit responsible for controlling the components of the system 100. The computing platform 110 is configured to process real-time data from other components, manage feedback loops for gigaseal formation, and run an AI framework or a machine learning model for data analysis. The computing platform 110 is further configured to monitor and control system operations, including sealing condition adjustments and recording data analysis. The computing platform 110 may be implemented as a computer, a workstation, or a server, and is equipped with network communication capabilities to enable data exchange, remote access, and integration with external databases or cloud-based analysis platforms.

The amplifier 112 is configured to amplify small electrical signals recorded from bath environment and ion channels during the patch clamp recording. The amplifier 112 enhances the signal strength, making the signals distinguishable and suitable for analysis, enabling real-time monitoring of the patch clamp recording and measurement of ion channel activity.

The digitizer 114 is configured to convert analog signals from the amplifier 112 into digital data and transmit the digital data to the computing platform 110 for further recording and analysis. By converting the signals into a digital format, the digitizer 114 enables the computing platform 110 to read and process the data, thereby fulfilling its purpose of facilitating high-quality digitized recordings of ion channel activity, which can be analyzed using the AI framework or the machine learning model. The DAQ system 116 is configured to provide real-time feedback to the computing platform 110, enabling real-time monitoring and control of patch clamp operations and parameters.

The computing platform 110 collaborates with the digitizer 114 and the DAQ system 116 to establish a feedback loop in the patch clamp system 100 for adjusting the pipette position, pressure, and sealing conditions based on real-time data, optimizing performance throughout the patch clamp recording.

The digitizer 114 and DAQ system 116 can be similar instruments, both responsible for signal acquisition and conversion. However, they may be configured as separate units to achieve specific purposes, such as handling different signal types or improving data resolution. When used together, they can complement each other by distributing processing loads for enhancing synchronization. Alternatively, they can be integrated into a single device, combining their functions to streamline signal processing and system efficiency.

The microscope 118 provides real-time visual feedback, focusing on the cell dish 102 to assist in positioning components during electrophysiological measurement monitoring. The microscope 118 transmits collected visual data to the computing platform 110, which uses this information to guide micromanipulator adjustments and optimize pipette-cell membrane interactions.

The micromanipulator 120 is configured to control components of the system 100, such as the pipette. For example, the micromanipulator 120 can position the pipette 126 relative to the cell membrane in the cell dish 102 based on real-time signals from the computing platform 110. The micromanipulator 120 receives control instructions for nanoscale adjustments, allowing pipette 126 positioning to establish stable lipid-glass seal formation with the cell membrane.

The headstage 122 secures the pipette 126 and serves as the interface for signal transmission. The headstage 122 relays the electrical signals detected by the recording electrodes 124 to the amplifier 112 while also functioning as the conduit for negative pressure applied by the pressure regulator 130 (e.g., pump). The headstage 122 maintains stable signal integrity between the patch clamp setup and the amplifier 112, enabling high-fidelity electrophysiological recordings.

The recording electrodes 124 are connected to the headstage 132. One of the recording electrodes 124 is positioned inside the pipette 126, while the another one of the recording electrodes 124 is placed in the bath solution within the cell dish 102.

The recording electrodes 124 are configured to detect ionic currents flowing through the bath environment and ion channels during the patch clamp recording. The detected signals are transmitted through the headstage 122 to the amplifier 112 and subsequently sent to the computing platform 110 for analysis. Feedback derived from the recording electrodes 124 is used for parameter adjustments, including pipette positioning and suction control.

The pipette 126 is a glass microtube that is connected to the headstage 130 and is permitted to make contact with the cell membrane in the cell dish 102, forming a gigaseal through suction applied by the pressure regulator 130. The positioning of pipette 126 is controlled by the micromanipulator 120 based on feedback from the microscope 118 and electrophysiological signals monitored by the computing platform 110. The pipette 126 facilitates stable gigaseal formation, minimizing leakage currents and improving the accuracy of ion channel activity measurements.

The pressure regulator 130 is coupled with the computing platform 110 and is configured to regulate the pressure applied to the pipette 126 to achieve and maintain a stable seal between the pipette 126 and the cell membrane within the cell dish 102. The pressure regulator 130 receives dynamic control signals from the computing platform 110, enabling real-time adjustments based on feedback from other sources, such as detection results from the recording electrodes 124. Controlling suction in this manner facilitates the formation of a reliable gigaseal.

The above configuration is set up for performing the patch clamp process to achieve unattended electrophysiological measurements.

Briefly, when performing the patch clamp process, the micromanipulator 120 positions the pipette 126 near the cell membrane in the cell dish 102. The pressure regulator 130 controls the pressure applied to the pipette 126, allowing the configuration to form a stable gigaseal and proceed with recording. The recording electrodes 124 detect ionic currents flowing through the ion channels during the patch clamp recording. The detected electrical signals are transmitted to the headstage 122 which then relays the signals to the amplifier 112, where the small ionic currents are amplified to improve the signal-to-noise ratio. The digitizer 114 and the DAQ system 116 convert the collected signals into digital data. The computing platform 110 processes this digital data, calculates the optimal seal condition, and drives the micromanipulator 120 for recording. The data is then analyzed using the AI framework or the machine learning of the computing platform 110.

In one embodiment, the computing platform's AI framework or machine learning model is trained using a structured training dataset. The training process begins with data preprocessing, where recordings undergo anomaly detection using K-nearest neighbors (KNN) to filter out recordings that exhibit inconsistent ion channel behavior. The remaining valid recordings are then segmented into different phases (e.g., rising, sustaining, and falling) to enhance feature extraction. The model of the computing platform 110 is trained using a 1D convolutional neural network (1DCNN) to capture spatial correlations, followed by a bidirectional long short-term memory (BiLSTM) network to learn long-term temporal dependencies in electrophysiological signals. During the training, an attention mechanism is incorporated to prioritize the most informative electrophysiological features. Throughout training, iterative optimization and validation are performed using labeled datasets, such that the model of the computing platform 110 effectively classifies ion channel types and characterizes their activation and inactivation kinetics.

Once the configuration is set up, meaning the components are positioned and activated for operation, the system executes a series of automated steps to achieve gigaseal formation, signal acquisition, and data analysis.

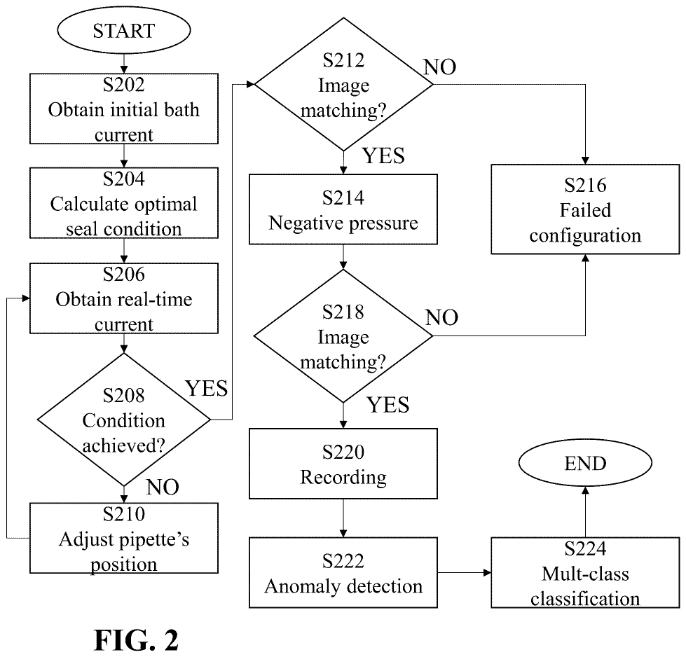

FIG. 2 illustrates a workflow of the automated patch clamp system 100 detailing the process for conducting electrophysiological recordings and analyzing ion channel kinetics according to some embodiments of the present invention. The process starts by initializing all hardware components and then verifies that all the necessary components are correctly installed and ready for the measurement. Specifically, the process includes step S202, S204, S206, S208, S210, S212, S214, S216, S218, S220, S222, S224,

In step S202, once the pipette 126 is immersed in the bath environment within the cell dish 102, the recording electrodes 124 detect the initial bath current or series resistance. The headstage 122 transmits the measured signals to the amplifier 112 for amplification, after which the digitizer 114 and the DAQ system 116 convert the amplified signals into digital form for processing by the computing platform 110.

In step S204 and step S206, the computing platform 110 monitors the electrical signal in real-time (e.g., real-time current) to determine whether the pipette 126 is aligned with a target cell (e.g., cell membrane) within the cell dish 102 under optimal sealing conditions. In step S208, if the determination does not match the optimal sealing condition, the process moves to step S210, where the micromanipulator 120 adjusts the pipette's position to correctly align with the target cell again. If the determination matches the optimal sealing condition, the process moves to step S212. In embodiment, the optimal sealing condition is determined through data-driven analysis and real-time feedback control. The computing platform 110 monitors the initial bath current or series resistance, and when the resistance reaches a predefined threshold (e.g., >1 GΩ), the computing platform 110 determines that the sealing condition is optimal.

In step S212, the microscope 118 provides visual feedback to verify that the pipette 126 is properly aligned for making contact with the cell membrane. If the visual feedback confirms that the pipette 126 is correctly positioned, the computing platform 110 generates a signal representing image matching based on the visual feedback and proceeds to step S214. If not, the process moves to step S216, where the computing platform generates a warning signal indicating a failed configuration.

In step S214, the pressure regulator 130 applies controlled pressure to the pipette 126 as it approaches the optimal sealing position. Simultaneously, the computing platform 110 monitors the pressure to regulate the suction level, which is advantageous to the formation of a proper lipid-glass seal.

In step S218, the computing platform 110 continues to monitor and adjust the applied pressure to the target cell until a stable gigaseal is formed based on real-time feedback electrical signal (e.g., generated by using the recording electrodes). If the computing platform 110 determines a stable gigaseal is formed, the process moves to step S220. If not, the process moves to step S216, where the computing platform 110 generates a warning signal indicating a failed configuration.

In step S220, after a gigaseal is formed and determined to be stable, the computing platform 110 switches to a recording mode, allowing for the measurement of ion channel activity in the cell membrane. Specifically, the measured signals are amplified by the amplifier 112 and sent to the digitizer 114 the DAQ system 116 for conversion from analog to digital format. The data containing the measured signals is then sent to the computing platform 110, where the computing platform 110's AI framework or machine learning model begins analyzing the signals.

In step S222, the computing platform 110 executes the AI framework or machine learning model for machine learning-based anomaly detection. The machine learning-based anomaly detection eliminates invalid recordings, including aberrant transient ion channel behaviors, failed recordings, noise distorted data, partially abnormal ion channel activity, and combinations thereof.

Next, in step S224, the computing platform 110 executes multi-class classification, namely, deep learning classification. During this process, ion channel kinetics are classified using a deep learning algorithm by the computing platform, including ion channel behavior, activation, and inactivation patterns. The computing platform generates a report summarizing the measurement results, which includes details of ion channel activity, their classification, and the overall electrophysiological properties observed during the measurement. The report can be provided in a digital format and is readable for human interpretation.

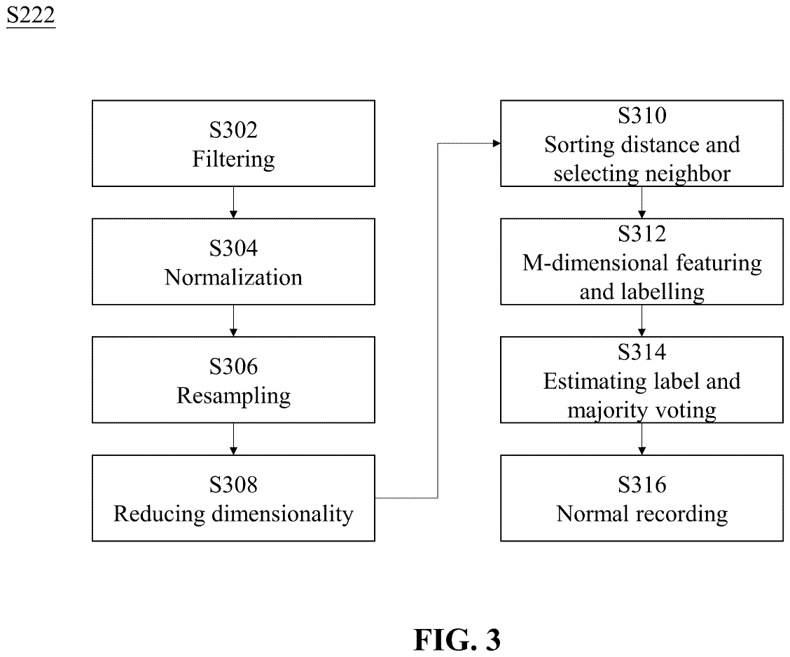

FIG. 3 illustrates a workflow of the machine learning-based anomaly detection of step S222 according to some embodiments of the present invention. Briefly, machine learning-based anomaly detection is achieved through signal feature processing, containing a systematic sequence of steps: filtering, normalization, resampling, and detection. Specifically, the process includes step S302, S304, S306, S308, S310, S312, S314, and S316.

The process begins with step S302. In step S302, a filtering process is performed to clean a raw patch clamp recording by removing unwanted noise, including electronic interference and biological variability. Otherwise, the noise obscures the true signal representing ion channel activity. The computing platform 110 may include a filter for signal processing, which is configured to smooth the signal while preserving its essential features, such as the peaks and troughs that correspond to ion channel openings and closings.

In step S304, the filtered patch clamp recording undergoes a normalization process to standardize it to a consistent scale, allowing the features of ion channel activity to be accurately captured. The computing platform 110 may include a normalization module configured to adjust the amplitude of the patch clamp recording signal to fit within a specified range or distribution, reducing biases.

In step S306, the patch clamp recording is downsampled to simplify processing or upsampled to capture more detailed temporal dynamics, making its temporal resolution compatible with detection requirements. The computing platform 110 may include a feature extractor, which performs feature extraction on the patch clamp recording, including amplitude (scale of ion channel activity), frequency components (periodic characteristics of ion channel opening and closing), and temporal patterns (time-based features).

In step S308, the extracted features are used to reduce the dimensionality of the patch clamp recording, which is to allow the machine learning model to focus on the most relevant aspects of the signal that may indicate anomalies.

In step S310, the computing platform's machine learning model calculates the distances among points and sorts them in ascending order. The machine learning model then selects the closest neighbors to the query point based on the smallest distance.

In step S312, the computing platform's machine learning model processes each feature as a vector in an M-dimensional space, where M represents different attributes or characteristics. The machine learning model analyzes these feature vectors and assigns a label that classifies the feature as either normal or anomalous.

In step S314, the computing platform's machine learning model selects K-nearest neighbors based on proximity in the feature space to estimate the label of an unlabeled recording. The label is determined through majority voting, maintaining consistency with the classification of its nearest neighbors.

Steps S310, S312, and S314 work together to enable anomaly detection by leveraging machine learning-based pattern recognition. The collaborative process allows the computing platform to generalize patterns from existing labeled data and detect deviations, effectively identifying anomalies in ion channel recordings.

In step S316, normal recording is achieved using machine learning mechanism, so as to reduce reliance on predefined thresholds and improving classification accuracy through learned feature representations. The machine learning mechanism maintain the quality and validity of the recorded electrophysiological data by identifying and filtering out any erroneous or physiologically irrelevant signals during the patch clamp process.

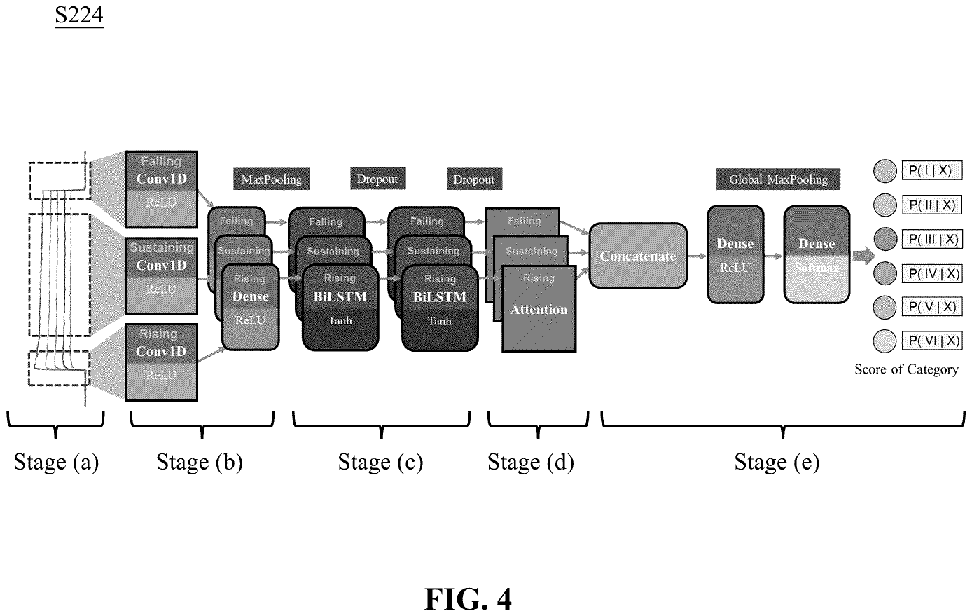

FIG. 4 shows a neural network architecture of deep learning for multi-class classification of step S224 according to some embodiments of the present invention. The multi-class classification of step S224 includes stage (a), (b), (c), (d), (e).
Stage (a) is segmentation of the recording based on dynamics. The raw electrophysiological recording is segmented into three distinct phases, rising, sustaining, and falling, based on the temporal dynamics of ion channel responses. The segmentation allows the model to analyze each phase independently, capturing transient changes in ion channel behavior. The segmented signals serve as structured inputs for the subsequent feature extraction process (e.g., an extraction process to electrophysiological features).

Stage (b) is feature extraction using one-dimensional convolutional neural network (Conv1D). In one embodiment, the computing platform 110 applies a convolutional neural network (CNN) to feature extraction. The Conv1D extracts spatial features and correlations among multiple response currents, effectively identifying ion channel types and characterizing their dynamic properties. Each phase (rising, sustaining, and falling) is processed by a separate Conv1D layer, allowing phase-specific spatial patterns to be captured. In one embodiment, MaxPooling is applied to reduce dimensionality and retain essential features. The extracted features provide the foundation for understanding the unique temporal and spatial characteristics of ion channel activity, feeding structured information into subsequent temporal processing layers.
Stage (c) is temporal dependency learning via BiLSTM. The feature maps obtained from Conv1D are passed to BiLSTM networks, which are specialized for capturing long-term dependencies in sequential data. This stage integrates spatial, temporal, and contextual patterns, refining the characterization of voltage-dependent activation, inactivation kinetics, and response variability. The dual-directional processing of BiLSTM ensures that dependencies between different time points are captured, regardless of whether they occur earlier or later in the sequence. Dropout layers help prevent overfitting, ensuring robust generalization across different ion channel types.
Stage (d) is feature prioritization via attention mechanism. The outputs from BiLSTM are further processed by an attention mechanism, which assigns different importance weights to the extracted features. The attention mechanism selectively enhances the most informative temporal features relevant to ion channel classification while diminishing the impact of less critical information. By dynamically adjusting feature emphasis, the attention mechanism strengthens the model's ability to discriminate between similar ion channel types and enhances classification precision.
Stage (e) is feature integration and classification. The attention-weighted outputs from the three independent processing branches (rising, sustaining, and falling) are concatenated to form a unified representation of the recording. The aggregated feature set undergoes further processing through dense layers with ReLU activation, allowing for non-linearity and improved feature interactions. A Global Maxpooling step extracts the most prominent features, which are passed to the final Softmax classification layer. Stage (e) then assigns a probability distribution across multiple ion channel classes, generating P(I|X), P(II|X), . . . , P(VI|X) to determine the most likely ion channel type based on the given recording.

After stage (e), the final output includes a classified ion channel type with an associated probability score, a structured feature representation of ion channel kinetics, and processed electrophysiological data.

The following compares the technical features achievable by the technical solution provided in the present invention with the existing deficiencies.

### (1). Predictive Sealing Model for Optimal Patch Clamp Recordings

Traditional patch clamp techniques face significant challenges due to variations in pipette morphology and cellular environments. These factors make it difficult to achieve a consistent and reproducible seal between pipettes and the cell membrane under varying conditions. The lack of a quantifiable model to predict and control the sealing conditions results in unpredictable electrical currents and a low signal-to-noise ratio. This severely impacts the quality of the acquired data, often leading to insufficient recordings that fail to accurately represent the electrophysiological activity of the cell.

In the present invention, the automated patch clamp system addresses these challenges by introducing a sealing monitor, that dynamically evaluates and adjusts the real-time sealing conditions. It uses initial electrical signals (such as bath current and series resistance) to predict and maintain the optimal lipid-glass sealing necessary for high-fidelity recordings. By continuously monitoring and adjusting the seal, the system ensures that the signal quality remains high, thus significantly improving the validity and authenticity of the acquired electrophysiological data. This function is particularly beneficial in research settings where precise data is critical for understanding cellular behavior, such as in neuroscience, cardiology, and pharmacology. When studying the behaviors of ion channels, the system ensures that each experimental recording captures the true physiological response, free from noise and interference.

### (2). Standardized and Data-Driven Gigaseal Formation

Achieving a gigaseal, a high-resistance seal between the pipette and the cell membrane, is the critical step in patch clamp experiments. In conventional systems, the decision to stop the pipette engaging and initiate gigaseal formation is based on the operator's subjective judgment. Systems typically monitor the resistance or current levels, the thresholds from operators'experience decide when the pipette has achieved an appropriate level of contact with the cell membrane to start the suction required for gigaseal formation. Those thresholds may vary between operators or even between experiments, leading to inconsistent results. Without standardized protocols, these procedures are often labor-intensive, requiring expert skills to adjust pressure and position. As a result, the yield of successful gigaseal formations is low, and the quality of the acquired data is inconsistent, limiting the adaptability of the systems across different cell types and conditions.

In the present invention, a sealing strategy is introduced, which is guided by a lipid-glass sealing mechanism, using real-time electrical signals to automatically calculate the optimal conditions for gigaseal formation. The system's data-driven approach standardizes the gigaseal process, eliminating subjective decision-making and reducing operator dependence. It ensures that gigaseal formation is consistent, reproducible, and based on objective electrical parameters, such as initial bath current or series resistance. Unlike traditional methods that rely on operator experience, the provided system uses the derived seal ratio from the lipid-glass interaction to control the entire gigaseal process, reducing the risk of seal failure or instability. The provided system continuously tracks changes in bath current and series resistance, automatically adjusting the pipette's position and the applied suction based on these objective electrical parameters. This sealing strategy ensures that the sealing process is not only optimized but also dynamically adjusted in real-time to maintain ideal conditions for gigaseal formation. This automated and standardized gigaseal formation process is particularly valuable in screening environments, such as pharmaceutical drug discovery, where large numbers of cells need to be tested in a consistent and reproducible manner. It significantly reduces the labor required for each experiment, increases success rates, and enhances data quality across a wide range of cellular environments.

### (3). Comprehensive Integration of Data Acquisition and Anomaly Recording Detection

Existing automated patch clamp systems focus primarily on data acquisition, leaving the analysis of ion channel activity to manual, post-experiment processes. This separation between data acquisition and analysis is time-consuming and inefficient. Additionally, these systems lack the ability to assess the validity of the acquired data during measurement, often resulting in poor-quality recordings that must be manually discarded or reanalyzed. This creates a bottleneck in the research workflow, reducing overall efficiency and throughput.

The provided system integrates automated data acquisition with comprehensive anomaly recording detection. Using built-in machine learning algorithms, the system evaluates the validity of the data as it is being recorded, detecting issues such as noise, drift, or failed recordings. The system can also automatically identify recordings, providing insights into the functional electrophysiological properties of the cells being studied. This integrated acquisition and detection design enhances the scope and efficiency of patch clamp experiments, enabling operators to quickly identify recordings, without the need for manual intervention. This integrated approach significantly speeds up the experimental process, providing rapid and actionable insights into cellular responses, enabling operators to make real-time adjustments or decisions based on immediate results.

### (4). Automated Multi-Class Ion Channel Kinetics Classification with Deep Learning

Traditional electrophysiological analysis is limited by its inability to simultaneously analyze multiple ion channels or classify them based on their kinetic behaviors during a single experiment. The manual interpretation of ion channel data is often slow and subjective, with operators needing to sift through large amounts of raw data to identify ion channel behaviors and characteristics. Moreover, manual methods are not well-suited for detecting complex temporal patterns in the data, especially when multiple ion channels are activated or inactivated simultaneously.

The provided system incorporates a deep learning model within the artificial intelligence framework to analyze whole-cell recordings. This deep learning model uses a combination of one-dimension convolutional neural networks, bidirectional long short-term memory networks and attention mechanism to capture both the spatial and temporal dynamics of ion channel behaviors. The system automatically classifies multiple ion channel types and tracks their activity across different time periods, distinguishing between activation, inactivation, and sustained states. The inclusion of an attention mechanism ensures that the system focuses on the most relevant parts of the data, providing highly accurate and detailed insights into ion channel kinetics. This function is ideal for multi-channel electrophysiological research, where the goal is to simultaneously monitor the activity of multiple ion channels in response to stimuli, drugs, or genetic modifications. For example, in neuroscience research, where understanding the interplay of different ion channels during neuronal firing is critical, the provided system enables operators to study these interactions in real-time, providing insights that would be impossible to obtain using traditional methods. The automated multi-class classification also allows operators to handle large datasets efficiently, making it suitable for high-throughput studies.

The advantages of the present invention are outlined as follows.

### (1). Tailored and Data-Driven Lipid-Glass Sealing Mechanism for Optimal Gigaseal Formation

Traditional patch clamp techniques and current automated systems are highly dependent on the operator's experience to determine when the pipette has sufficiently interacted with the cell membrane to begin gigaseal formation. Manual observation of electrical signals like bath current or series resistance is often subjective, leading to variability in the formation of high-quality seals. The inability to control or predict the precise interaction between the pipette and cell membrane (the contact gap) means that the formation of the gigaseal is often inconsistent. Current automated systems merely replicate manual methods, lacking any real improvement in terms of success rates or data quality.

In the present invention, a lipid-glass sealing strategy is introduced, which applies real-time electrical signal monitoring and lipid-glass sealing mechanisms to optimize the pipette-cell interaction, automatically adjusting key parameters for each experiment. The provided system tailors sealing conditions for each test using a derived seal ratio, ensuring that each gigaseal is formed at the optimal moment based on real-time analysis of the bath current and series resistance. The provided system also significantly increases the gigaseal formation success rate, as it eliminates operator guesswork and replaces it with data-driven, automated adjustments. By offering nanoscale precision in pipette positioning and suction application, the provided system ensures optimal high-resistance seals, which are essential for minimizing leakage current, enhancing signal-to-noise ratio, and ensuring reproducibility and data quality.

### (2). First System to Integrate Recording Acquisition with Recording Analysis

The majority of automated patch clamp systems on the market are limited to data acquisition and are incapable of recording analysis. Once the data is acquired, operators must manually inspect the recordings to evaluate data validity, ion channel behavior, and cellular characteristics. This manual approach is time-consuming, prone to human error, and often requires highly trained personnel. Moreover, traditional systems lack the capability to perform kinetic analysis or classify ion channel types during the experiment.

The provided can integrate data acquisition with recording analysis. It automatically identifies recordings, determines ion channel types and behaviors, and performs kinetic characterization. Detect and eliminate data anomalies (e.g., signal artifacts or failed recordings) using machine learning algorithms like K-nearest neighbors, ensuring that only high-quality, physiologically valid data is remained for further ion channel kinetics analysis. The deep learning-based multi-class classification provides an immediate evaluation of electrophysiological properties and cellular functions during the experiment, saving time and reducing the need for post-experiment kinetics analysis.

### (3). Automated Anomaly Detection for Data Integrity

Current automated patch clamp systems focus solely on data acquisition, lacking components for anomaly detection or data quality assessment during recordings. As a result, operators must manually sift through raw data to identify aberrant signals, transient ion channel behaviors, or failures in recording integrity, a process prone to human error that can lead to wasted time and misinterpretation of results.

In the present invention, the provided system integrates machine learning-based anomaly detection, continuously monitoring acquired data for potential issues such as transient ion channel behaviors without physiological relevance, artifacts or noise from mechanical disturbances, and failed recordings that could skew data analysis. By using algorithms like K-nearest neighbors, the system flags and removes these anomalies, ensuring the final dataset accurately reflects true biological activity. For example, in pharmacological research, where ion channel recordings are used to assess drug efficacy, the ability to detect and exclude anomalous data ensures that only meaningful, high-quality recordings are used, significantly enhancing the reliability of conclusions drawn from the data.

### (4). Multi-Class Classification of Ion Channel Activity Using Deep Learning

Current systems are limited in their ability to classify ion channel behavior and analyze multiple ion channels kinetics. Manual analysis is typically restricted to one ion channel or phase at a time, with post-experiment processing taking hours or even days. Moreover, traditional methods fail to leverage deep learning to detect complex patterns in ion channel behavior, such as subtle changes in activation and inactivation kinetics.

In the present invention, the provided system employs a deep learning framework that integrates one-dimension convolutional neural networks, bidirectional long short-term memory and attention mechanism to classify multiple ion channel kinetics. This approach captures non-linear temporal and spatial relationships between different ion channels, uses attention mechanisms to focus on the most critical parts of the electrophysiological signal, and provides classification of ion channel activation and inactivation dynamics. The provided system enables operators to monitor and classify the behavior of multiple ion channels simultaneously, offering valuable insights into channel interactions and conditions in single patch clamp system.

### (5). High Success Rates and Data Yield through Automation and Integrated Control

While automated systems exist, they often rely on traditional manual pipette-cell membrane interaction techniques, offering limited improvements in gigaseal success rates and data yields. Additionally, the lack of integrated control and real-time lipid-glass sealing monitor in these systems results in inconsistent outcomes, requiring frequent manual intervention.

In the present invention, the provided system combines real-time lipid-glass sealing feedback, automated sealing control, and machine learning-based anomaly detection, deep learning-driven data analysis to achieve high gigaseal success rates by automating the sealing process and optimizing conditions for each test, thereby minimizing failures due to human error. This approach increases data yield by reducing the number of failed or poor-quality recordings, enhancing overall experimental efficiency. Moreover, the provided system reduces operator dependence by enabling fully unattended operation, from pipette positioning to data analysis, allowing operators to run more experiments with minimal supervision. In high-throughput environments like biotech and pharmaceutical labs, where thousands of experiments must be conducted efficiently, the system's ability to consistently deliver high-quality data with minimal human intervention significantly boosts productivity and cost-effectiveness.

The functional units and modules of the apparatuses and methods in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes executing in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can be included, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. An automated patch clamp system, comprising:
a computing platform;
a cell dish configured to hold at least one cell in a physiological solution for electrophysiological measurement;
a pipette configured to interface with a cell membrane of the cell to establish a gigaseal;
a micromanipulator configured to position the pipette relative to the cell membrane based on control signals from the computing platform;
a pressure regulator coupled to the pipette and configured to regulate a suction pressure applied for gigaseal formation;
a pair of recording electrodes, wherein one of the recording electrodes is positioned within the pipette and another one is in a bath solution within the cell dish, and wherein the recording electrodes are configured to detect at least one ionic current flowing through ion channels of the cell;
a digitizer configured to convert the detected ionic current into digitized electrophysiological signals;
wherein the computing platform is further configured to process the digitized electrophysiological signals by executing an artificial intelligence (AI) framework or a machine learning model for ion channel kinetics analysis and anomaly detection, classifying ion channel types based on electrophysiological features extracted from the ion channel kinetics analysis.

2. The automated patch clamp system according to claim 1, wherein the computing platform establishes a feedback loop using real-time measurement data from the digitizer to adjust the pipette position and the suction pressure.

3. The automated patch clamp system of claim 2, further comprising a microscope configured to provide real-time visual feedback to the computing platform for guiding the micromanipulator's positioning of the pipette.

4. The automated patch clamp system of claim 1, wherein the computing platform applies the AI framework or the machine learning model for multi-class classification in the ion channel kinetics analysis, classifying ion channel kinetics into ion channel behavior, activation, and inactivation patterns.

5. The automated patch clamp system of claim 1, wherein the AI framework or the machine learning model applies machine learning-based anomaly detection to identify and filter out invalid recordings, containing noise distortions, physiologically irrelevant signals, and combinations thereof.

6. The automated patch clamp system of claim 5, wherein the computing platform processes the extracted electrophysiological features as M-dimensional feature vectors and applies a K-nearest neighbors (KNN) algorithm for classification of the machine learning-based anomaly detection.

7. The automated patch clamp system of claim 1, wherein the computing platform applies a bidirectional long short-term memory (BiLSTM) network to ion channel kinetics analysis to process feature maps obtained from a one-dimensional convolutional neural network (Conv1D), capturing long-term dependencies in sequential data.

8. The automated patch clamp system of claim 7, wherein the computing platform applies an attention mechanism within the AI framework of the machine learning model to prioritize the most informative electrophysiological features for ion channel classification by assigning different importance weights to extracted features.

9. The automated patch clamp system of claim 8, wherein the computing platform integrates attention-weighted outputs from multiple processing branches, including rising, sustaining, and falling phases, through concatenation to generate a probability distribution across multiple ion channel classes.

10. The automated patch clamp system of claim 1, wherein the computing platform generates a human-readable report summarizing ion channel activity, classification results, and electrophysiological properties in a digital format.

11. An automated patch clamp method, comprising:
holding at least one cell in a physiological solution using a cell dish for electrophysiological measurement;
interfacing with a cell membrane of the cell to establish a gigaseal using a pipette;
positioning the pipette relative to the cell membrane using a micromanipulator based on control signals from a computing platform;
coupling a pressure regulator to the pipette to regulate a suction pressure applied for gigaseal formation;
detecting at least one ionic current flowing through ion channels of the cell using a pair of recording electrodes;
converting the detected ionic current into digitized electrophysiological signals using a digitizer; and
processing the digitized electrophysiological signals by executing an artificial intelligence (AI) framework or a machine learning model, using the computing platform, for ion channel kinetics analysis and anomaly detection, thereby classifying ion channel types based on electrophysiological features extracted from the ion channel kinetics analysis.

12. The automated patch clamp method according to claim 11, further comprising:
establishing a feedback loop using real-time measurement data from the digitizer to adjust the pipette position and the suction pressure.

13. The automated patch clamp method of claim 12, further comprising:
providing real-time visual feedback using a microscope to the computing platform for guiding the micromanipulator's positioning of the pipette.

14. The automated patch clamp method of claim 11, further comprising:
applying the AI framework or the machine learning model for multi-class classification in the ion channel kinetics analysis, thereby classifying ion channel kinetics into ion channel behavior, activation, and inactivation patterns.

15. The automated patch clamp method of claim 11, further comprising:
applying machine learning-based anomaly detection to identify and filter out invalid recordings, containing noise distortions, physiologically irrelevant signals, and combinations thereof.

16. The automated patch clamp method of claim 15, further comprising:
processing the extracted electrophysiological features as M-dimensional feature vectors and applying a K-nearest neighbors (KNN) algorithm for classification of the machine learning-based anomaly detection.

17. The automated patch clamp method of claim 11, further comprising:
applying a bidirectional long short-term memory (BiLSTM) network to ion channel kinetics analysis to process feature maps obtained from a one-dimensional convolutional neural network (Conv1D), thereby capturing long-term dependencies in sequential data.

18. The automated patch clamp method of claim 17, further comprising:
applying an attention mechanism within the AI framework of the machine learning model to prioritize the most informative electrophysiological features for ion channel classification by assigning different importance weights to extracted features.

19. The automated patch clamp method of claim 18, further comprising:
integrating attention-weighted outputs from multiple processing branches, which includes rising, sustaining, and falling phases, through concatenation to generate a probability distribution across multiple ion channel classes.

20. The automated patch clamp method of claim 1, further comprising:
generating a human-readable report summarizing ion channel activity, classification results, and electrophysiological properties in a digital format.
